(19) United States
(12) Patent Application Publication
(10) Pub. No.: US 2025/0144148 A1
(43) Pub. Date: May 8, 2025

(54) METHOD FOR PREPARING CELLS FOR TREATING DISEASES, METHOD FOR PROMOTING MIGRATION ABILITY OF CELLS, METHOD FOR PROMOTING PROLIFERATION ABILITY OF CELLS, AND CELLS OBTAINED BY THESE METHODS

(71) Applicants: ROHTO PHARMACEUTICAL CO., LTD., Osaka (JP); SONIKURE HOLDINGS LIMITED, Hong Kong (CN)

(72) Inventors: Hikari KUROGI, Osaka (JP); Smriti ARYA, Hong Kong (CN); Ka Ming CHOI, Hong Kong (CN); Hau Yi Paulisally LO, Hong Kong (CN); Wai Leung Langston SUEN, Hong Kong (CN); Wai Ki LAU, Hong Kong (CN); Jamison Thomas ARMSTRONG, Hong Kong (CN)

(73) Assignees: ROHTO PHARMACEUTICAL CO., LTD., Osaka (JP); SONIKURE HOLDINGS LIMITED, Hong Kong (CN)

(21) Appl. No.: 18/834,375

(22) PCT Filed: Jan. 26, 2023
(86) PCT No.: PCT/JP2023/002483

§ 371 Date: Jul. 30, 2024

(30) Foreign Application Priority Data
Feb. 3, 2022 (JP) 2022-015966

(51) Int. Cl. A61K 35/28; C12N 5/0775; C12N 13/00

(52) U.S. Cl. CPC A61K 35/28; C12N 5/0667; C12N 13/00

(57) ABSTRACT

An object of the present invention is to provide a method for obtaining cells having a high migration ability or cells having a high proliferation ability as cells having an increased therapeutic effect on diseases. The present invention is a method for preparing cells for treating diseases, a method for promoting a migration ability of cells, and a method for promoting a proliferative ability of cells, each of the methods comprising subjecting cells to ultrasonic treatment. The frequency of the ultrasonic treatment is preferably 30 kilohertz to 2.0 megahertz, and the cells are preferably mesenchymal stem cells. In addition, the present invention also includes cells obtained by the preparation method of the present invention.

### TECHNICAL FIELD

The present invention relates to a method for preparing cells for treating diseases, a method for promoting the migration ability of cells, a method for promoting the proliferation ability of cells, and cells obtained by these methods.

### BACKGROUND ART

Mesenchymal stem cells (MSCs) are progenitor cells having pluripotency, which were first isolated from bone marrow by Friedenstein in 1982 (see Non Patent Document 1). It has been shown that mesenchymal stem cells are present in various tissues such as bone marrow, umbilical cord, and adipose, and mesenchymal stem cell transplantation is expected as a new treatment method for various intractable diseases (see Non Patent Documents 1 and 2). Recently, it has been known that cells having comparable function are present in stromal cells of adipose tissue, a placenta, umbilical cord, an egg membrane, or the like. Because of this, mesenchymal stem cells are sometimes also referred to as mesenchymal stromal cells.

It is well known that mesenchymal stem cells are used in regenerative medicine or the like because they have a tissue repairing action and an immunosuppressive action. However, the mechanism by which mesenchymal stem cells exert such effects has not yet been fully elucidated. Recently, it has been reported that mesenchymal stem cells have the properties of actively accumulating in an inflammatory tissue or a tumor tissue in the body, and it is considered that mesenchymal stem cells may secrete a cytokine, an exosome, or the like in an inflammatory tissue or a tumor tissue in the body to act on surrounding cells and thereby promote the repair of the inflammatory tissue. (Non Patent Documents 3 and 4). Under such circumstances, there is a demand for a technique for further enhancing the therapeutic effect of mesenchymal stem cells.

### CITATION LIST

### Non Patent Literature

NPL 1: Pittenger F. M. et al. Science, (1999), 284, pp. 143-147
PTL 2: Cell Transplantation, (2016), 25, pp. 829-848
PTL 3: iScience, (2019) 15, pp. 421-438
PTL 4: Int J Med Sci. (2018), 15 (10), pp. 1051-1061

### SUMMARY OF INVENTION

### Technical Problem

Under the circumstances described above, an object of the present invention is to provide a method for obtaining cells having a high migration ability or cells having a high proliferation ability as cells having a further enhanced therapeutic effect on diseases.

### Solution to Problem

The present inventors have considered that increasing the accumulation of cells in an inflammatory tissue or a tumor tissue leads to a further enhanced therapeutic effect of the cells, and have conducted intensive research in order to solve the above problem. As a result, the present inventors have found that ultrasonic treatment of mesenchymal stem (stromal) cells (MSCs) is effective in enhancing the migration ability and the proliferation ability of the mesenchymal stem (stromal) cells, and completed the present invention. According to the present invention, it is possible to provide cells having a high migration ability and cells having a high proliferation ability. That is, a summary of the present invention is as follows.
[1] A method for preparing cells for treating diseases comprising subjecting cells to ultrasonic treatment.
[2] The method for preparing cells for treating diseases according to [1], wherein a frequency of the ultrasonic treatment is 30 kilohertz to 2.0 megahertz.
[3] The method for preparing cells for treating diseases according to [1] or [2], wherein the cells are mesenchymal stem cells.
[4] A method for promoting a migration ability of cells comprising subjecting cells to ultrasonic treatment.
[5] The method for promoting a migration ability of cells according to [4], wherein a frequency of the ultrasonic treatment is 30 kilohertz to 2.0 megahertz.
[6] The method for promoting a migration ability of cells according to [4] or [5], wherein the cells are mesenchymal stem cells.
[7] A method for promoting a proliferation ability of cells comprising subjecting cells to ultrasonic treatment.
[8] The method for promoting a proliferation ability of cells according to [7], wherein a frequency of the ultrasonic treatment is 30 kilohertz to 2.0 megahertz.
[9] The method for promoting a migration ability of cells according to [7] or [8], wherein the cells are mesenchymal stem cells.
[10] Cells treated with an ultrasonic wave having a frequency of 30 kilohertz to 2.0 megahertz.
[11] Mesenchymal stem cells treated with an ultrasonic wave having a frequency of 30 kilohertz to 2.0 megahertz.

### Advantageous Effects of Invention

According to the present invention, it is possible to provide a novel method for promoting the migration ability of cells, and cells having a migration ability promoted thereby. In addition, according to the present invention, it is possible to provide a novel method for promoting the proliferation ability of cells, and cells having a proliferation ability promoted thereby. According to the present invention, cells having an excellent migration ability and cells having an excellent proliferation ability can be obtained, and thus it is considered that a large number of cells can be accumulated in a disease site such as an inflammatory tissue or a tumor tissue and that the therapeutic effect of the cells on diseases can be enhanced.

### BRIEF DESCRIPTION OF DRAWINGS

FIG. 1 is a diagram showing the cell proliferation ability of mesenchymal stem cells.

FIG. 2 is a diagram showing the cell proliferation ability of mesenchymal stem cells.

FIG. 3 is a diagram showing the cell proliferation ability of mesenchymal stem cells.

FIG. 4 is a diagram showing the cell proliferation ability of mesenchymal stem cells.

FIG. 5 is a diagram showing the cell proliferation ability of mesenchymal stem cells.

FIG. 6 shows results of a scratch assay.

FIG. 7 is a diagram showing a quantification of results of a scratch assay.

FIG. 8 shows staining of cells migrating to the lower surface of a Transwell in a migration ability test.

FIG. 9 shows the number of cells migrating to the lower surface of a Transwell in a migration ability test.

FIG. 10 shows staining of cells migrating to the lower surface of a Transwell in a migration ability test.

FIG. 11 shows the number of cells migrating to the lower surface of a Transwell in a migration ability test.

FIG. 12 shows results of repair over time in a scratch assay.

FIG. 13 is a diagram showing quantified repair rates (%) after 9 hours in a scratch assay.

FIG. 14 shows staining of cells migrating to the lower surface of a Transwell in a migration ability test.

FIG. 15 shows the number of cells migrating to the lower surface of a Transwell in a migration ability test.

FIG. 16 shows results of repair over time in a scratch assay.

FIG. 17 is a diagram showing quantified repair rates (%) after 9 hours in a scratch assay.

### DESCRIPTION OF EMBODIMENTS

Hereinafter, the method for preparing cells for treating diseases, the method for promoting the migration ability of cells, the method for promoting the proliferation ability of cells, and the cells obtained by these methods according to the present invention will be described in detail.

### Method for Preparing Cells for Treating Diseases

The method for preparing cells for treating diseases according to the present invention is characterized by subjecting cells to ultrasonic treatment. According to the present invention, by subjecting cells to ultrasonic treatment under a specific condition, the migration ability of the cells can be enhanced, and cells having high accumulation in a disease site such as an inflammatory tissue or a tumor tissue can be provided, and thus the therapeutic effect on diseases can be further enhanced. Hereinafter, the method for preparing cells for treating diseases according to the present invention will be described.

### Cells

The cells in the present invention are not particularly limited as long as they are cells exerting an effect on the treatment of a disease, and examples thereof include mesenchymal stem cells, placenta-derived stem cells, umbilical cord blood-derived cells, peripheral blood mononuclear cells (including neutrophils, eosinophils, basophils, lymphocytes, monocytes and the like), red blood cells, T cells, NK cells, NKT cells, NKM cells, LAK cells, dendritic cells, fibroblasts, hematopoietic stem cells, iPS cells, ES cells, bone marrow cells, myocardial cells, hepatocytes, nerve cells, dermal cells, adipose cells, and other cells that constitute each tissue. Among these, mesenchymal stem cells, placenta-derived stem cells, umbilical cord blood-derived cells, peripheral blood mononuclear cells, and bone marrow cells are preferable and mesenchymal stem cells are more preferable, from the viewpoint of further improving the migration ability by ultrasonic treatment and exerting an excellent effect on disease treatment.

In the present invention, the mesenchymal stem cells mean cells that have the potency to differentiate into one or more types of cells belonging to the mesenchymal system (bone cells, cardiac muscle cells, chondrocytes, tendon cells, adipose cells, or the like), and can proliferate while maintaining such a potency. The term mesenchymal stem cells used in the present invention mean the same cells as mesenchymal stromal cells, and there is no particular distinction between the two. In addition, the mesenchymal stem cells are sometimes also simply referred to as mesenchymal cells. Examples of a tissue containing mesenchymal stem cells include adipose tissue, umbilical cord, bone marrow, umbilical cord blood, endometrium, placenta, amnion, chorion, decidua, dermis, skeletal muscle, periosteum, dental follicle, periodontal ligament, dental pulp, and tooth germ. For example, adipose tissue-derived mesenchymal stem cells mean mesenchymal stem cells contained in adipose tissue, and may also be referred to as adipose tissue-derived mesenchymal stromal cells. Among these, adipose tissue-derived mesenchymal stem cells, umbilical cord-derived mesenchymal stem cells, bone marrow-derived mesenchymal stem cells, placenta-derived mesenchymal stem cells, and dental pulp-derived mesenchymal stem cells are preferable, adipose tissue-derived mesenchymal stem cells and umbilical cord-derived mesenchymal stem cells are more preferable, and adipose-derived mesenchymal stem cells are most preferable, from the viewpoint of effectiveness in treating a disease, from the viewpoint of availability, or the like.

The mesenchymal stem cells in the present invention may be, for example, cells provided by PromoCell GmbH, Lonza Group AG, Biological Industries, Veritas Corporation, R&D Systems, Inc., Corning Incorporated, or the like, or may be cells that can be prepared by a method well known to those skilled in the art. In addition, the mesenchymal stem cells may be primary cells isolated from a donor tissue, or may be cells of an established cell line.

As a preparation method well known to those skilled in the art, for example, the mesenchymal stem cells of the present invention can be prepared by obtaining cells from each tissue by enzymatic digestion or physical cutting, then sedimenting the cells, resuspending the cells in an appropriate medium, and culturing the cells. In addition, examples of a method for culturing cells includes, but are not limited to, a method for culturing cells on a solid surface of a petri dish, a flask, or the like, a method of agitating and culturing sphere-like cell masses by agglomerating cells, and a method for culturing cells by stirring the cells adhered onto a micro carrier.

Examples of a species of the mesenchymal stem cells in the present invention include a human, a horse, a cow, a sheep, a pig, a dog, a cat, a rabbit, a mouse, and a rat.

In the present invention, tissue-derived mesenchymal stem cells such as adipose tissue-derived mesenchymal stem cells, umbilical cord-derived mesenchymal stem cells, or bone marrow-derived mesenchymal stem cells refer to any cell population including tissue-derived mesenchymal stem cells such as adipose tissue-derived mesenchymal stem cells, umbilical cord-derived mesenchymal stem cells, or bone marrow-derived mesenchymal stem cells, respectively. At least 20% or more, preferably 30%, 40%, 50%, 60%, 70%, 75%, 80%, 85%, 90%, 93%, 96%, 97%, 98%, or 99%, of the cell population consists of tissue-derived mesenchymal stem cells such as adipose tissue-derived mesenchymal stem cells, umbilical cord-derived mesenchymal stem cells, or bone marrow-derived mesenchymal stem cells.

The medium used for culturing the cells of the present invention is not particularly limited as long as it is a medium that can culture mesenchymal stem cells, and such a medium may be manufactured by supplementing a basal medium with a serum, and/or with one or more serum substitutes such as albumin, transferrin, a fatty acid, insulin, sodium selenite, cholesterol, a collagen precursor, a trace element, 2-mercaptoethanol, or 3′-thiolglycerol. Such a medium may, as necessary, be further supplemented with a substance such as a lipid, an amino acid, a protein, a polysaccharide, a vitamin, a growth factor, a low-molecular-weight compound, an antibiotic, an antioxidant, pyruvic acid, a buffering agent, or an inorganic salt.

Examples of the basal medium include an IMDM medium, a Medium 199 medium, an Eagle's Minimum Essential Medium medium (EMEM), a MEM-α medium, a Dulbecco's modified Eagle's Medium medium (DMEM), a Ham's F12 medium, an RPMI 1640 medium, a Fischer's medium, an MCDB201 medium, and mixed media thereof.

Examples of the serum include, but are not limited to, human serum, fetal bovine serum (FBS), bovine serum, calf serum, goat serum, horse serum, pig serum, sheep serum, rabbit serum, rat serum and the like. When a serum is used, a basal medium may be supplemented therewith in an amount of 5 v/v % to 15 v/v %, preferably 10 v/v %.

The mesenchymal stem cells in the present invention may appropriately be cells subjected to repeated cryopreservation and thawing. In the present invention, cryopreservation can be carried out by suspending mesenchymal stem cells in a cryopreservation solution well known to those skilled in the art and cooling the same. The thawed mesenchymal stem cells may be appropriately cultured before the next cryopreservation. The culture of the mesenchymal stem cells is carried out using the above medium that can culture mesenchymal stem cells, and is not particularly limited.

The mesenchymal stem cells in the present invention can be prepared as described above, and may be defined as cells having the following properties;
(1) they exhibit adhesion to plastic under culture conditions in a standard medium,
(2) they are positive for surface antigens CD44, CD73, and CD90 and negative for CD31, CD34, and CD45, and
(3) they can differentiate into bone cells, adipose cells, or cartilage cells under culture conditions.

The mesenchymal stem cells in the present invention can be used for various diseases, and specific diseases include GVHD, chondrolysis, rheumatoid arthritis, systemic lupus erythematosus, psoriatic arthritis, spondylarthritis, osteoarthritis, gout, psoriasis, multiple sclerosis, amyotrophic lateral sclerosis, Alzheimer's disease, Parkinson's disease, congestive heart failure, stroke, aortic stenosis, renal failure, nephrotic syndrome, uremia, lupus, pancreatitis, allergy, fibrosis, anemia, atherosclerosis, restenosis, chemotherapy/radiation-associated complication, type I diabetes, type II diabetes, hepatic failure, autoimmune hepatitis, hepatitis C, primary biliary cirrhosis, primary sclerosing cholangitis, fulminant hepatitis, celiac disease, nonspecific colitis, intestinal lymphangiectasia, protein-losing enteropathy, Crohn's disease, allergic conjunctivitis, diabetic retinopathy, Sjogren's syndrome, atopic disease, uveitis, allergic rhinitis, food allergy, anaphylaxis, autoimmune disease, drug hypersensitivity, mastocytosis, asthma, asbestos, silicosis, chronic obstructive pulmonary disease, chronic granulomatous inflammation, cystic fibrosis, histiocytosis, sarcoidosis, glomerulonephritis, vasculitis, dermatitis, HIV-associated cachexia, cerebral malaria, ankylosing spondylitis, leprosy, COPD, pulmonary fibrosis, fibromyalgia, cancer such as esophageal cancer, gastroesophageal reflux disease, Barrett's esophagus, aplastic anemia, graft-versus-host disease, sickle cell disease, CVID, hyper-IgM syndrome, IgA deficiency, transient hypogammaglobulinemia, X-linked anhema gammaglobulinemia, chronic mucocutaneous candidiasis, DiGeorge's syndrome, X-linked lymphoproliferative syndrome, ataxia telangiectasia, cartilage-hair hypoplasia, combined immunodeficiency, hyper-IgE syndrome, MHC deficiency, severe combined immunodeficiency, Wiskott-Aldrich syndrome, Chediak-Higashi syndrome, chronic granulomatous disease, leukocyte adhesion deficiency, IFN-γ receptor deficiency, interleukin (IL)-12 deficiency, IL-12 receptor β1 deficiency, ZAP-70 deficiency, angioedema and the like.

The placenta-derived stem cells in the present invention are cells that are fetal-derived, have low immunogenicity, and have the ability to differentiate into triploblastic cells. Examples of the placenta-derived stem cells include, but are not limited to, mesenchymal stem cells, hematopoietic stem cells, neural stem cells, placental pluripotent cells, embryonic-like stem cells ant the like.

The umbilical cord blood-derived cells in the present invention are cells included in umbilical cord blood, and examples thereof include hematopoietic stem cells, mononuclear cells, B cells, T cells, NK cells, platelets and the like.

The peripheral blood mononuclear cells in the present invention are mononuclear cells isolated from peripheral blood, and examples thereof include B cells, T cells, NK cells, monocytes, dendritic cells and the like.

Examples of the bone marrow cells in the present invention include hematopoietic stem cells and mesenchymal stem cells.

### Ultrasonic Wave (Ultrasonic)

In the present invention, the “ultrasonic wave (ultrasonic)” refers to a sound wave having a frequency above the human audible range, and refers to a sound wave having a high frequency of 20 kHz (kilohertz) or more. In the present invention, there is no particular limitation on the ultrasonic wave (ultrasonic), and the frequency thereof is, for example, 20 kHz (kilohertz) to 20 MHz (megahertz), preferably 20 kHz to 10 MHz, more preferably 20 kHz to 5 MHz, further preferably 20 kHz to 3 MHz, particularly preferably 30 kHz to 2 MHz, more particularly preferably 40 kHz to 1.5 MHz and most preferably 500 kHz to 1.5 MHz.

In the present invention, the intensity of the ultrasonic wave (ultrasonic) is not particularly limited, and is, for example, 0.1 mW/cm² to 200 mW/cm², preferably 0.6 mW/cm² to 100 mW/cm², further preferably 1 mW/cm² to 75 mW/cm², and more preferably 10 mW/cm² to 50 mW/cm².

In the present invention, the ultrasonic duty cycle is not particularly limited, and is, for example, 1% to 95%, preferably 5% to 75%, more preferably 10% to 60%, and further preferably 20% to 50%.

In the present invention, the ultrasonic treatment time is appropriately adjusted according to the frequency, the intensity, the duty cycle, or the like, and is, for example, 10 seconds to 1 hour, preferably 30 seconds to 30 minutes, more preferably 1 minute to 20 minutes, more preferably 1.5 minutes to 15 minutes, and particularly preferably 2 minutes to 10 minutes.

In the present invention, when the cells are mesenchymal stem cells, the conditions for ultrasonic treatment are preferably a frequency of 20 kHz to 3 MHz, an intensity of 0.6 mW/cm² to 100 mW/cm², a duty cycle of 5% to 75%, and a treatment time of 1 minute to 20 minutes, more preferably a frequency of 30 kHz to 2 MHz, an intensity of 1 mW/cm² to 75 mW/cm², a duty cycle of 10% to 60%, a treatment time of 1.5 minutes to 15 minutes, and further preferably a frequency of 40 kHz to 1.5 MHz, an intensity of 10 mW/cm² to 50 mW/cm², a duty cycle of 20% to 50%, and a treatment time of 2 minutes to 10 minutes.

The ultrasonic generator that can be used in the present invention is not particularly limited as long as it can generate an ultrasonic wave, and examples thereof include METRON ACCUSONIC PLUS ULTRASOUND THERAPY UNIT (MODEL AP100, Metron Medical Australia Pty Ltd).

When cells are subjected to ultrasonic treatment in the present invention, a culture vessel such as a flask or a dish or a tube containing a cell suspension may be set in an ultrasonic apparatus for the treatment, or an ultrasonic wave may be directly applied by inserting an ultrasonic source into a culture vessel or a cell suspension. That is, in the present invention, the ultrasonic treatment of cells may be indirect irradiation via a culture vessel or the like in which the cells are cultured, or direct irradiation of the cells themselves or a cell suspension. In the present invention, it is preferable to set the culture vessel in an ultrasonic apparatus for treatment, from the viewpoint of the effects of the present invention.

One embodiment of the method for preparing cells for treating diseases according to the present invention will be specifically described.

Cells are seeded in a culture vessel and cultured, and after 0 days to 4 days, preferably 1 day to 2 days of culture, the culture vessel is set in an ultrasonic generator in the state of the cells being still cultured, and ultrasonic treatment is carried out from the bottom surface of the culture vessel. The frequency, the intensity, the duty cycle, and the treatment time can be appropriately adjusted according to the cell type, the number of cells, the purpose, or the like, and the ultrasonic treatment is carried out under the ranges of conditions described above. The state of the cells to be ultrasonically treated may be, for example, the state of being cultured in a culture vessel as described above, the state of cultured cells being collected in a test tube or the like, or the state of frozen cells being thawed.

The cells after ultrasonic treatment may be used for disease treatment as they are, or may be used for disease treatment after being cultured for a certain period of time. In addition, the cells after ultrasonic treatment may be cultured and then passaged one or more times before being used for disease treatment.

The cells obtained by the method for preparing cells for treating diseases according to the present invention have a higher migration ability than usual cells and also have a wound repair ability, as indicated by a scratch assay, superior to that of usual cells. Therefore, according to the present invention, it is possible to provide cells having high accumulation in a disease site, thereby further enhancing the therapeutic effect on an inflammatory disease, a tumor, or the like. In particular, when the cells obtained by the method for preparing cells for treating diseases according to the present invention are mesenchymal stem cells, the therapeutic effect can be further enhanced in the treatment of various diseases for which the above mesenchymal stem cells can be used. The cells prepared by the method of the present invention are preferably used as a medicament for treatment of diseases.

### Method for Promoting Migration Ability of Cells

The present invention also includes a method for promoting the migration ability of cells, wherein the method comprises subjecting cells to ultrasonic treatment. According to the present invention, by subjecting cells to ultrasonic treatment under a specific condition, it is possible to promote the migration ability of the cells and increase the accumulation of the cells in a disease site. Because of this, the cells obtained by the method of the present invention can be preferably used for treating a disease such as an inflammatory disease or a tumor. Specific details of the method for promoting the migration ability of cells according to the present invention are the same as those of the above method for preparing cells for treating diseases according to the present invention, and thus to a specific description of the former, the description in the section of the method for preparing cells for treating diseases can be applied.

### Method for Promoting Proliferation Ability of Cells

The present invention also includes a method for promoting the proliferation ability of cells, the method comprising subjecting cells to ultrasonic treatment. According to the present invention, by subjecting cells to ultrasonic treatment under a specific condition, it is possible to promote the proliferation ability of the cells and accumulate a large number of such cells in a disease site. Because of this, the cells obtained by the method of the present invention can be preferably used for treating a disease such as an inflammatory disease or a tumor. Specific details of the method for promoting the proliferation ability of cells according to the present invention are the same as those of the above method for preparing cells for treating diseases according to the present invention, and thus to a specific description of the former, the description in the section of the method for preparing cells for treating diseases can be applied.

### Cells Obtained by the Method of the Present Invention

The methods of the present invention are the above method for preparing cells for treating diseases, the method for promoting the migration ability of cells, and the method for promoting the proliferation ability of cells, and the present invention also includes cells obtained by these methods. The cells obtained by the method of the present invention are cells treated with an ultrasonic wave by the method of the present invention, and the cell type and the ultrasonic conditions (frequency, intensity, duty cycle, treatment time, and the like) are as described in the section of the method for preparing cells for treating diseases. Specific examples thereof include cells treated with an ultrasonic wave having a frequency of 40 kilohertz to 1.5 megahertz, and mesenchymal stem cells treated with an ultrasonic wave having a frequency of 40 kilohertz to 1.5 megahertz. The cells obtained by the method of the present invention are preferably used as a medicament for disease treatment. The present invention also includes inventions of a pharmaceutical composition for disease treatment, which contains the cells obtained by the method of the present invention.

The cells obtained by the method of the present invention are the cells having a promoted migration ability and proliferation ability and having an increased wound repair ability, as indicated by a scratch assay, as compared with usual cells, due to ultrasonic treatment under a specific condition, and it is not clear what change has occurred in the cells by ultrasonic treatment to obtain such cells. What has been at least clarified by the present invention is that the cells subjected to ultrasonic treatment under the above conditions are cells having a promoted migration ability and proliferation ability and having an enhanced wound repair ability, as indicated by a scratch assay, as compared with usual cells and are preferably used as cells for treating diseases. It is considered that cells are affected in many ways by ultrasonic treatment. It is considered that a combination of various property changes such as promotion of any protein synthesis, inhibition of any protein synthesis, deactivation or activation of a protein already present, or an influence on a cell membrane results in the exertion of the effects of promotion of the migration ability, promotion of the proliferation ability, and enhancement of the wound repair ability as indicated by a scratch assay. It is considered that it is impossible or impractical to specify all such property changes by a further study, measurement, or the like. Because of this, the cells of the present invention are specified by a method as cells obtained by the method for preparing cells for treating diseases, the method for promoting the migration ability of cells, or the method for promoting the proliferation ability of cells according to the present invention.

The present invention also includes a culture supernatant of the cells obtained by the method of the present invention. Specific examples thereof include a culture supernatant of cells treated with an ultrasonic wave having a frequency of 40 kilohertz to 1.5 megahertz, and a culture supernatant of mesenchymal stem cells treated with an ultrasonic wave having a frequency of 40 kilohertz to 1.5 megahertz. The culture supernatant of the cells obtained by the method of the present invention is preferably used for a therapeutic purpose against a disease, a cosmetic, a different purpose, or the like.

### EXAMPLES

Hereinafter, the present invention will be described in detail with reference to Examples and Test Examples, but the present invention is not limited by these Examples and the like.

### Test Example 1: Ultrasonic Treatment of Adipose-Derived Mesenchymal Stem Cells-1

Adipose-derived mesenchymal stem cells (hereinafter referred to as “ADMSCs”) were seeded in a 35 mm culture vessel at 6,000 cells/cm² using a medium for mesenchymal stem cells (Growth medium 2, PROMOCELL GmbH). After seeding, only on day 3 (one irradiation) or on day 2 and day 3 (two irradiations), the culture vessel in which the ADMSCs were cultured was set in an ultrasound device (manufactured by Opharmic), and ultrasonic treatment was carried out from the bottom surface of the culture vessel. The frequencies were 40 kilohertz (KHz) and 1 megahertz (MHz), and the intensity was 30 mW/cm². The duty cycle was 20%, and the treatment times were 2 minutes, 5 minutes, and 10 minutes. The control group was not treated with an ultrasonic wave (CNTL). The proliferation ability of the cells after ultrasonic treatment was evaluated. Cell passage and cell counting were carried out on day 4 after the final ultrasonic treatment. First, the culture supernatant of the cells was removed, the cells were washed with PBS, and TrpLE was added to detach the cells. After centrifugation, about 10 μL of the cells resuspended in a medium was collected, and cell counting was carried out using a trypan blue staining solution. After the counting, the cells were seeded in a fresh culture vessel.

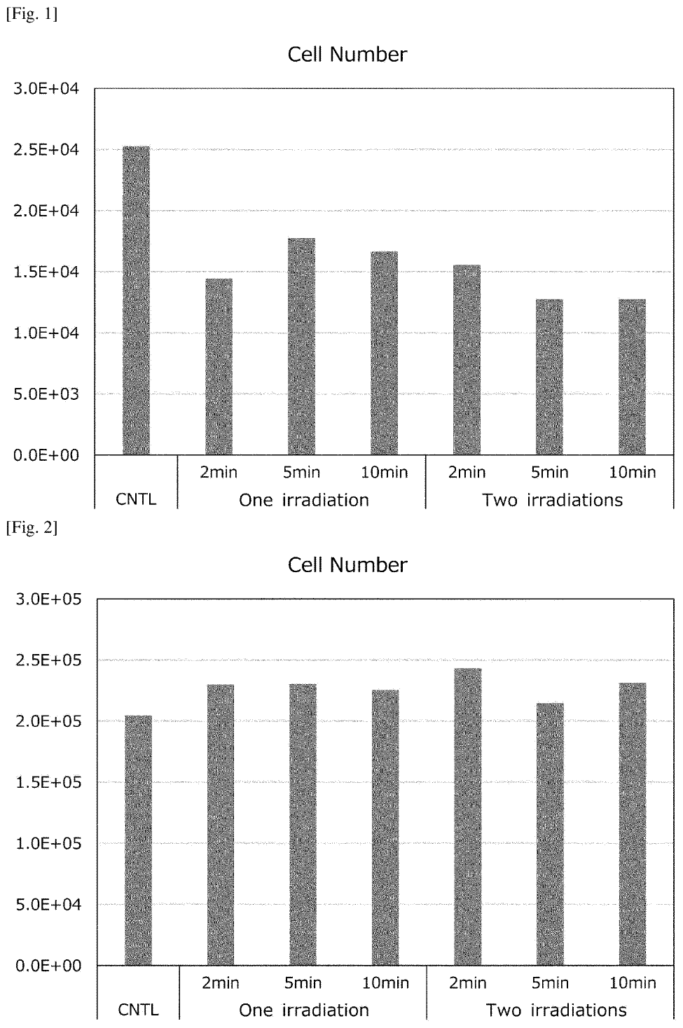

Although a decrease in proliferation ability was observed under any of the conditions at 40 kilohertz (KHz) (FIG. 1), an increase in proliferation ability was observed at 1 megahertz (MHz) (FIG. 2).

### Test Example 2: Ultrasonic Treatment of Adipose-Derived Mesenchymal Stem Cells-2

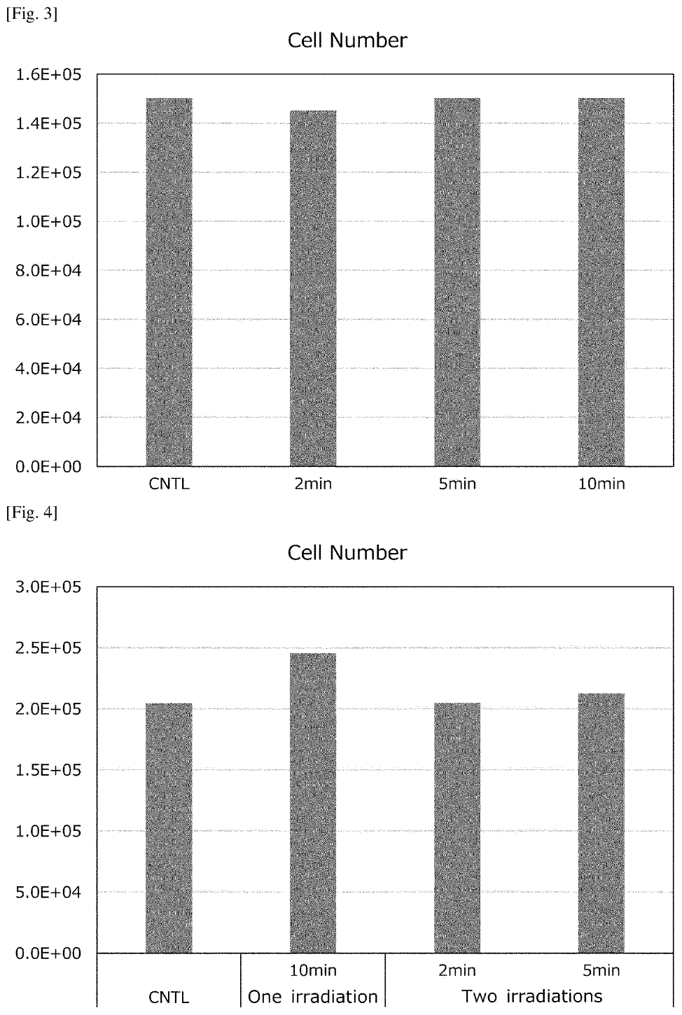

In the same manner as in Test Example 1, ADMSCs were cultured and subjected to ultrasonic treatment after 1 and 2 days. The frequency was 1.5 megahertz (MHz), the intensity was 30 mW/cm², the duty cycle was 20%, and the treatment times were 2 minutes, 5 minutes, and 10 minutes. The negative control was not treated with an ultrasonic wave. The proliferation ability of the cells after ultrasonic treatment was evaluated in the same manner as in Test Example 1. Results of counting the number of cells are shown in FIG. 3.

As shown in FIG. 3, no influence on proliferation ability was observed at 1.5 megahertz (MHz).

### Test Example 3: Ultrasonic Treatment of Adipose-Derived Mesenchymal Stem Cells-3

In the same manner as in Test Example 1, ADMSCs were cultured and subjected to ultrasonic treatment after 1 day (one irradiation) or both after 1 day and after 2 days (two irradiations). The frequency was 1 megahertz (MHz), the intensity was 30 mW/cm², the duty cycle was 50%, and the treatment time was 10 minutes for the one-irradiation group whereas the treatment times was 2 minutes and 5 minutes for the two-irradiation group. The negative control was not treated with an ultrasonic wave. The proliferation ability of the cells after ultrasonic treatment was evaluated in the same manner as in Test Example 1. Cell counting results are shown in FIG. 4.

As shown in FIG. 4, an increase in proliferation ability was observed in both irradiations.

### Test Example 4: Ultrasonic Treatment of Adipose-Derived Mesenchymal Stem Cells-4

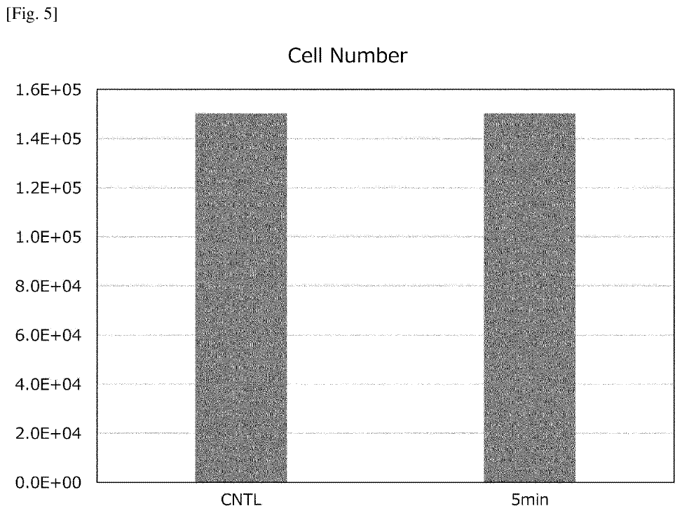

In the same manner as in Test Example 1, ADMSCs were cultured and subjected to ultrasonic treatment on day 1 and day 2 twice in total when the day of seeding was set to day 0. The frequency was 1.5 megahertz (MHz), the intensity was 30 mW/cm², the duty cycle was 50%, and the treatment time was 10 minutes. The negative control was not treated with an ultrasonic wave. The proliferation ability of the cells after ultrasonic treatment (day 3 after seeding) was evaluated in the same manner as in Test Example 1. The results of counting the number of cells are shown in FIG. 5.

As shown in FIG. 5, no influence on proliferation ability was observed even at 1.5 megahertz (MHz) and a duty cycle of 50%.

### Test Example 5: Scratch Assay for ADMSCs Subjected to Ultrasonic Treatment

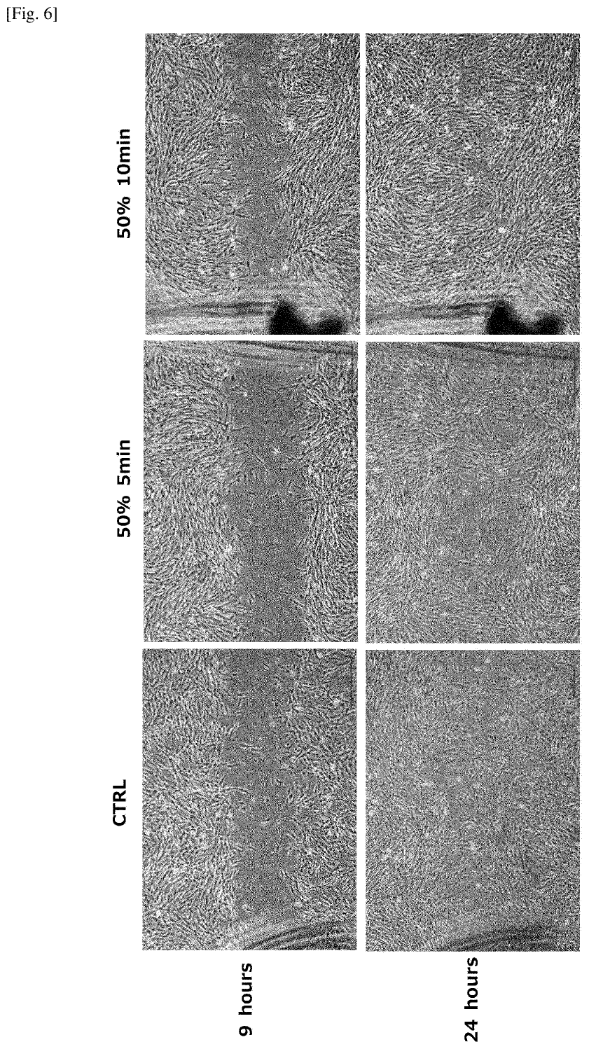

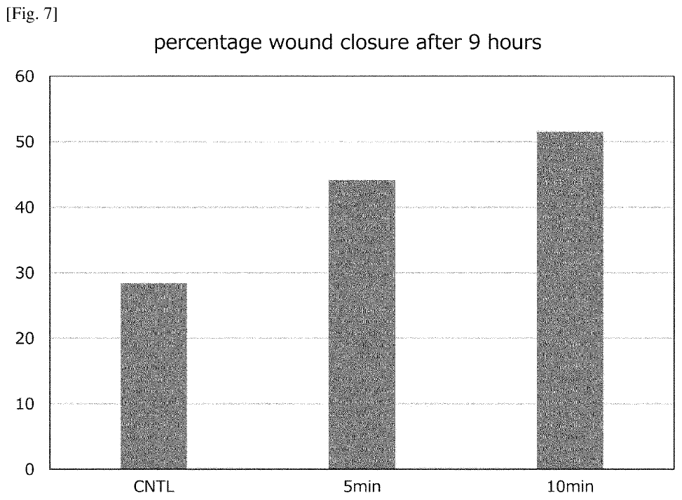

In the same manner as in Test Example 1, ADMSCs were cultured and subjected to ultrasonic treatment on day 1 and day 2 twice in total when the seeding date was set to day 0. The frequency was 1.5 megahertz (MHz), the intensity was 30 mW/cm², the duty cycle was 50%, and the treatment times were 5 minutes and 10 minutes. The ADMSCs subjected to ultrasonic treatment were seeded in a culture vessel having a diameter of 35 mm at 15,000 cells/cm² using a medium for mesenchymal stem cells (PROMOCELL GmbH). After confirming that the cells were in a confluent state, the center of the culture vessel in which the ADMSCs were cultured was scratched in a straight line using a 200 μL pipette tip to create a gap. After that, the cells were washed twice with PBS, 2 mL of a GM2 medium (PROMOCELL GmbH) was added, and the cells were cultured at 37° C. and a carbon dioxide concentration of 5%. 0, 4, 9, and 24 hours after the creation of the gap, the cells were observed under a microscope and images were acquired. After 9 hours, the width of the gap for each cell was measured. Results are shown in FIG. 6 and FIG. 7.

As shown in FIG. 6 and FIG. 7, the ultrasonic treatment enhanced the degree of wound repair as indicated by the scratch assay.

### Test Example 6: Migration Ability Test of ADMSCs Subjected to Ultrasonic Treatment

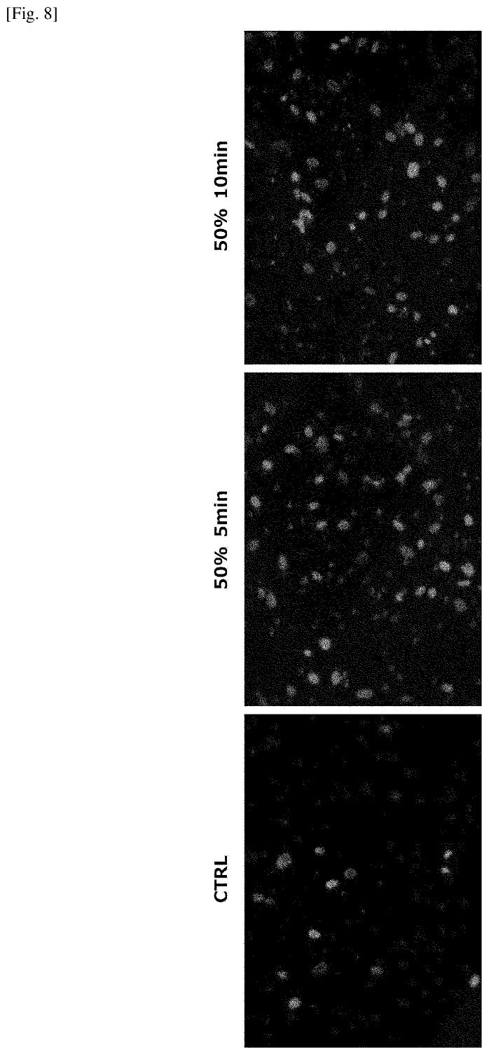

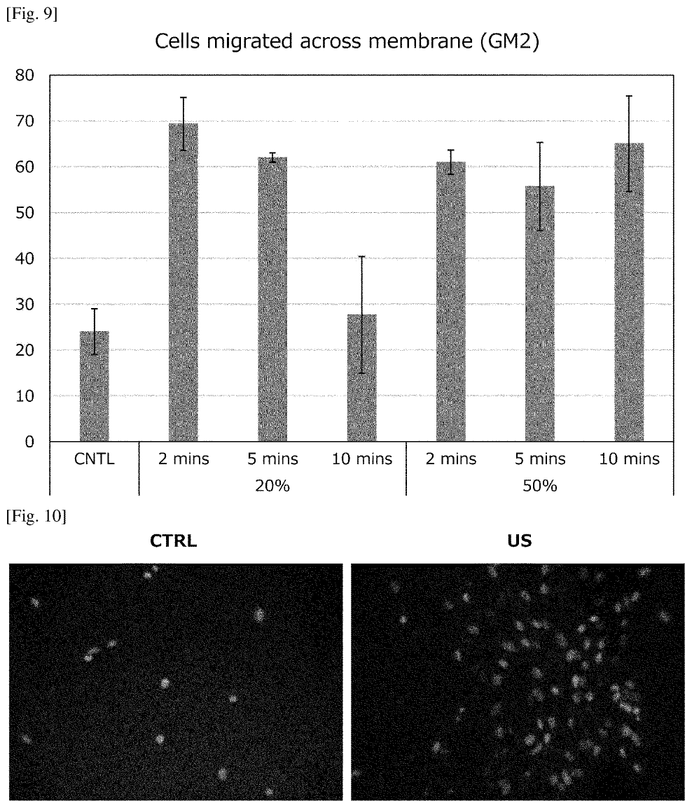

In the same manner as in Test Example 1, ADMSCs were cultured, and the ADMSCs subjected to ultrasonic treatment on day 1 and day 2 twice in total when the seeding date was set to day 0 were seeded at 3,000 cells each in a Transwell culture vessel (Corning) for a 24-well plate using a medium for mesenchymal stem cells (PROMOCELL GmbH). After 24 hours, the cells were washed with PBS, and 4% paraformaldehyde was added to fix the cells migrating to the lower surface of the Transwell. These cells were stained with DAPI. Cell counting was carried out under a fluorescence microscope, and the average values of results of three measurements were compared. In addition, fluorescence micrographs were also taken. In the ultrasonic treatment, the frequency was 1.5 megahertz (MHz), the intensity was 30 mW/cm², the duty cycle was 20% or 50%, and the treatment times were 2 minutes, 5 minutes, and 10 minutes. Results are shown in FIG. 8 and FIG. 9.

As shown in FIG. 8 and FIG. 9, under any of the irradiation conditions, the number of the cells migrating to the lower surface of the Transwell increased as compared with the control subjected to no irradiation, indicating that ultrasonic treatment enhanced the migration ability of the cells.

Although the data is not shown, an increase in the expression level of a migration ability-related gene by ultrasonic treatment was observed under any of the conditions, and a tendency to be high was observed particularly at a frequency of 1.5 megahertz (MHz).

### Test Example 8: Subculture of ADMSCs Subjected to Ultrasonic Treatment

ADMSCs subjected to ultrasonic treatment in the same manner as in Test Example 1 were detached using trypsin, subcultured for one passage, and cultured in a medium for mesenchymal stem cells (PROMOCELL GmbH) to obtain subcultured cells. The frequency of ultrasonic treatment was 1.5 megahertz (MHz), the intensity was 30 mW/cm², the duty cycle was 50%, and the treatment time was 5 minutes.

### Test Example 9: Migration Ability Test of Subcultured Cells

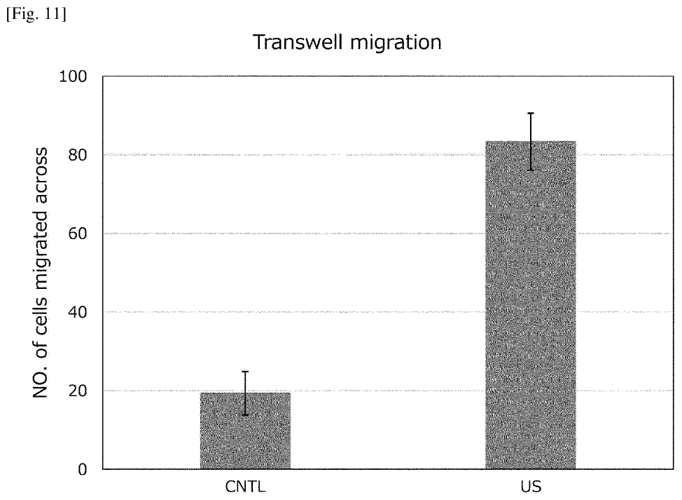

For the subcultured cells obtained in Test Example 8, a migration ability test was carried out in the same manner as in Test Example 6. After 24 hours, the cells migrating to the lower surface of the Transwell were counted, and the average values of results of three measurements were compared. In addition, fluorescence micrographs were also taken. Results are shown in FIG. 10 and FIG. 11. In addition, the ultrasonically untreated group is set forth as CNTL, and the ultrasonically treated group is set forth as US.

As shown in FIG. 10 and FIG. 11, even for the subcultured cells, ultrasonic treatment increased the number of cells migrating to the lower surface of the Transwell.

### Test Example 10: Scratch Assay of Subcultured Cells

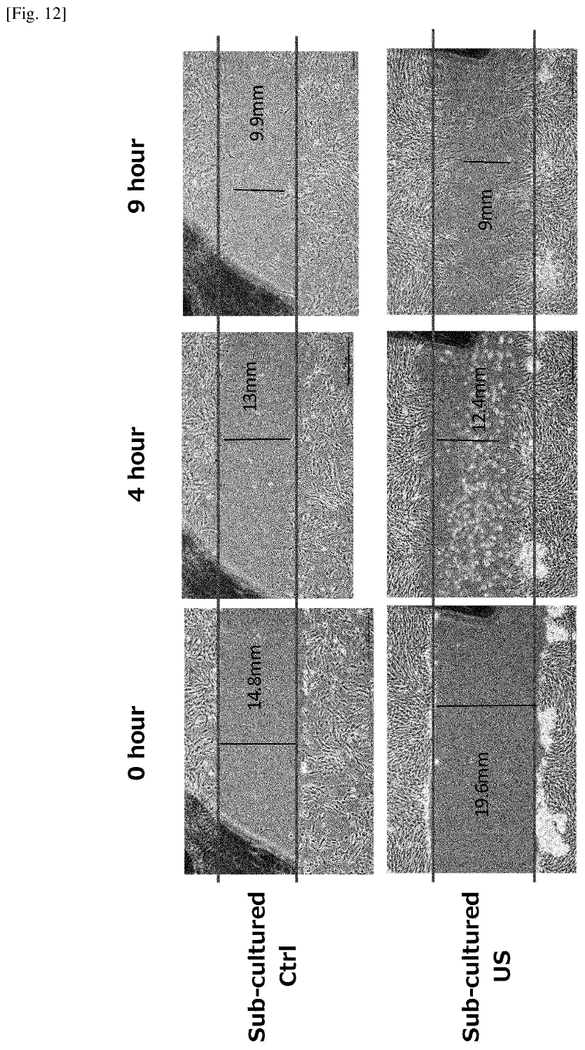

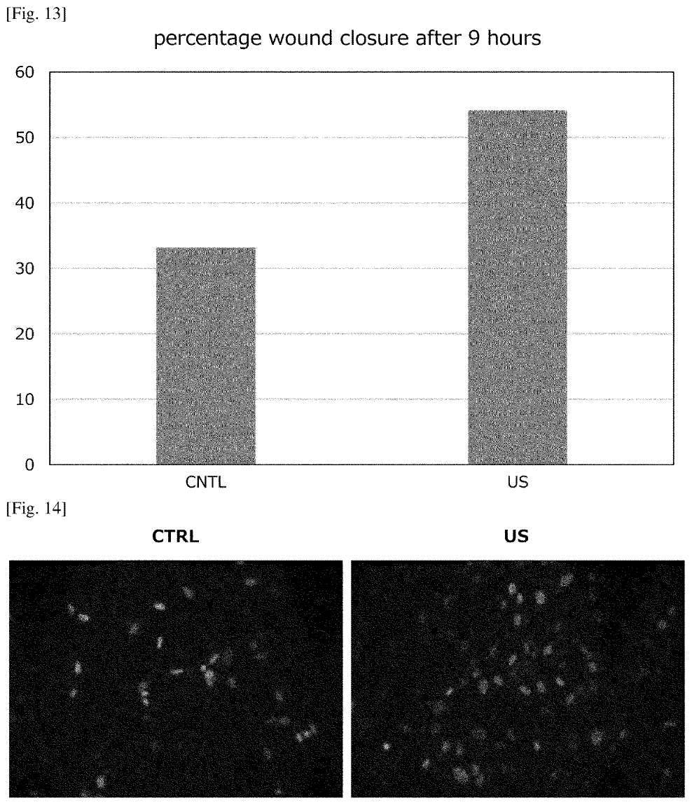

For the subcultured cells obtained in Test Example 8, in the same manner as in Test Example 5, the center of the culture vessel in which the ADMSCs were cultured was scratched in a straight line to create a gap, and 0, 4, 9, and 24 hours after the start of culture, the cells were observed under a microscope, and images were acquired. In addition, after 9 hours, the width of the gap for each cell was measured. Results are shown in FIG. 12 and FIG. 13. In addition, the ultrasonically untreated group is set forth as CNTL, and the ultrasonically treated group is set forth as US.

Even for the subcultured cells, ultrasonic treatment enhanced the degree of wound repair as indicated by the scratch assay (FIG. 12 and FIG. 13).

### Test Example 11: Freezing and Culture of ADMSCs Subjected to Ultrasonic Treatment

ADMSCs subjected to ultrasonic treatment in the same manner as in Test Example 8 were detached using trypsin and centrifuged to obtain a precipitation of the cells. The supernatant was removed, then an appropriate amount of a cell cryopreservation solution (Lonza Group AG) was added, and the cells were suspended. The resulting cell suspension solution was dispensed into cryotubes, then stored in the freezer at −80° C., then transferred to the gas phase above liquid nitrogen, and continued to be stored. The ultrasonically treated frozen cells were revived and cultured in a medium for mesenchymal stem cells (PromoCell GmbH) to obtain ultrasonically treated frozen cultured cells.

### Test Example 12: Migration Ability Test of Ultrasonically Treated Frozen Cultured Cells

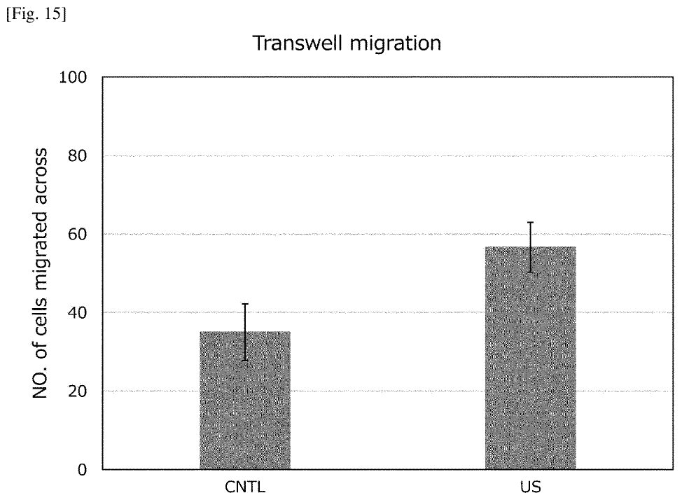

For the ultrasonically treated frozen cultured cells obtained in Test Example 11, a migration ability test was carried out in the same manner as in Test Example 6. That is, 24 hours after seeding in the Transwell culture vessel, the cells migrating to the lower surface of the Transwell were counted, and the average values of results of three measurements were compared. Results are shown in FIGS. 14 and 15.

In addition, the ultrasonically untreated group is set forth as CNTL, and the ultrasonically treated group is set forth as US.

Even for the frozen cultured cells, ultrasonic treatment increased the number of cells migrating to the lower surface of the Transwell as compared with the untreated frozen cultured cells.

### Test Example 13: Scratch Assay of Ultrasonically Treated Frozen Cultured Cells

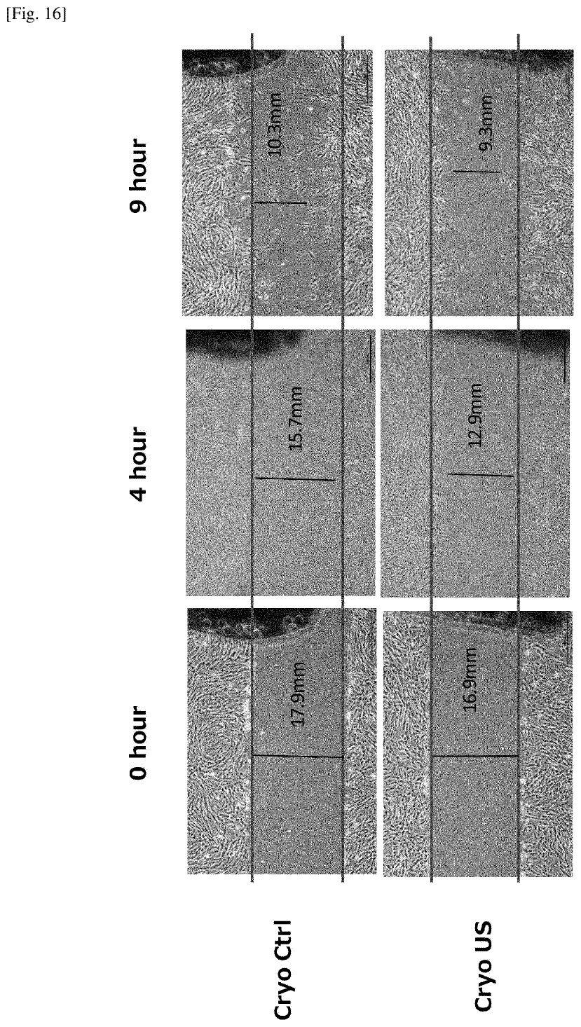

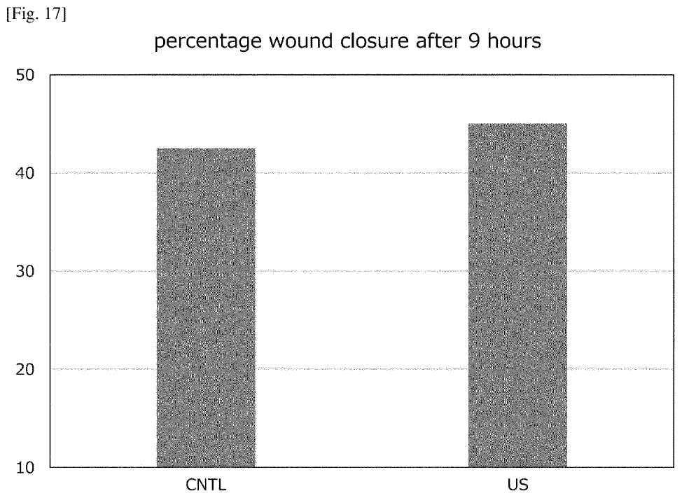

For the ultrasonically treated frozen cultured cells obtained in Test Example 11, in the same manner as in Test Example 5, the center of the culture vessel in which the cells were cultured was scratched in a straight line to create a gap, and 0, 4, 9, and 24 hours after the start of culture, the cells were observed under a microscope, and images were acquired. After 9 hours, the width of the gap for each cell was measured. Results are shown in FIG. 16 and FIG. 17. In addition, the ultrasonically untreated group is set forth as CNTL, and the ultrasonically treated group is set forth as US.

Even for the frozen cultured cells, ultrasonic treatment accelerated the migration rate of cells after scratching and the rate of wound repair as indicated by the scratch assay (FIG. 16 and FIG. 17).

### INDUSTRIAL APPLICABILITY

According to the present invention, it is possible to provide a novel method for promoting the migration ability of cells, and cells having a migration ability promoted thereby. In addition, according to the present invention, it is possible to provide a novel method for promoting the proliferation ability of cells, and cells having a proliferation ability promoted thereby. Therefore, according to the present invention, cells having an excellent migration ability and cells having a promoted proliferation ability can be obtained, and thus it is considered that a large number of cells can be accumulated in a disease site such as an inflammatory tissue

1. A method for preparing cells for treating diseases comprising subjecting cells to ultrasonic treatment.

2. The method for preparing cells for treating diseases according to claim 1, wherein a frequency of the ultrasonic treatment is 30 kilohertz to 2.0 megahertz.

3. The method for preparing cells for treating diseases according to claim 1, wherein the cells are mesenchymal stem cells.

4. A method for promoting a migration ability of cells comprising subjecting cells to ultrasonic treatment.

5. The method for promoting a migration ability of cells according to claim 4, wherein a frequency of the ultrasonic treatment is 30 kilohertz to 2.0 megahertz.

6. The method for promoting a migration ability of cells according to claim 4, wherein the cells are mesenchymal stem cells.

7. A method for promoting a proliferation of cells comprising subjecting cells to ultrasonic treatment.

8. The method for promoting a proliferation of cells according to claim 7, wherein a frequency of the ultrasonic treatment is 30 kilohertz to 2.0 megahertz.

9. The method for promoting a proliferation of cells according to claim 7, wherein the cells are mesenchymal stem cells.

10. Cells treated with an ultrasonic wave having a frequency of 30 kilohertz to 2.0 megahertz.

11. Mesenchymal stem cells treated with an ultrasonic wave having a frequency of 30 kilohertz to 2.0 megahertz.
